(19) United States
(12) Patent Application Publication
(10) Pub. No.: US 2025/0049672 A1
(43) Pub. Date: Feb. 13, 2025

(54) COMPOSITIONS AND METHODS FOR HAIR

(71) Applicant: L'OREAL, Paris (FR)

(72) Inventor: Kazumitsu KAWAKAMI, Westfield, NJ (US)

(73) Assignee: L'OREAL, Paris (FR)

(21) Appl. No.: 18/232,587

(22) Filed: Aug. 10, 2023

(51) Int. Cl. A61K 8/41; A61K 8/34; A61K 8/92; A61Q 5/12

(52) U.S. Cl. CPC A61K 8/416; A61K 8/342; A61K 8/922; A61Q 5/12; A61K 2800/5922

(57) ABSTRACT

The instant disclosure relates to compositions and methods for treating, caring for, and/or conditioning keratin fibers, such as hair. The compositions comprise a combination of at least one cationic esterquat compound, optionally at least one cationic compound other than an esterquat compound, at least one branched fatty alcohol, and at least one fatty ester. The methods comprise applying the compositions to keratin fibers, such as hair.

### FIELD OF THE DISCLOSURE

The present disclosure relates to compositions and methods for treating, caring for, and/or conditioning keratin fibers, such as hair.

### BACKGROUND

Many consumers desire to use cosmetic and care compositions that enhance the appearance of keratinous substrates such as hair, e.g., by changing the color, style, and/or shape of the hair, and/or by imparting various cosmetic properties to hair, such as shine and conditioning. However, hair can become dry or damaged for various reasons, e.g., weather exposure, poor nutrition, mechanical treatments (e.g. brushing hair), styling treatments using chemicals, dyeing, heat, nutrition, etc. Even cleansing products can remove hair's natural oils causing dryness, which can lead to a dull appearance and to split ends.

Chemical treatments include, for example, hair bleaching and coloring, permanents, waving products, and relaxing treatments (straightening treatments). These chemical treatments change the look of hair by changing its physical structure, which inevitably causes a certain degree of damage to the hair. Environmental factors, such as salt water, sunlight, and heat, are also known to damage hair. Damaged hair is characterized by unnatural changes to the protein structure of the individual hair strands or shafts. Damage results in split ends, dry straw-like hair, hair that is easily broken, and hair that is “frizzy” and unmanageable. Because the visible portion of hair is dead, it has no ability to regenerate itself. There are numerous over the counter and salon treatments that purport to repair damaged hair. These include conditioners, hot oil treatments, hydrolyzed proteins, vitamin formulations, and exotic fruit, leaf, or root extracts. These treatments, however, provide only limited improvement to the hair, Therefore, hair repair technologies that restore the properties of hair back to their natural level are desired.

The popularity and usage of oils for dry hair treatments has increased due to their effectiveness and simplicity. Commonly used oils include olive oil, mineral oil, avocado oil, apricot kernel oil, rice bran oil, and coconut oil. However, these treatments can leave the hair feeling greasy. In addition, the effects are not usually seen after more than several hours (e.g. 8 hours) of treatment and several treatments are usually required, making it time consuming and labor intensive.

In addition to improving frizz control and smoothness, consumers with curly or coily hair are especially in need of hair treatments that provide good curl definition without weighing down the hair. Thus, there is still a need for providing improved manageability of wet hair (e.g. improved wet combability and smoothness) and dry hair (e.g. improved hair alignment, reduced unwanted volume (especially reduced frizz), while increasing smoothness and moisture feel. In addition to providing such caring and conditioning benefits, there is also a need to develop hair care products that can impart other benefits simultaneously, such as improved styling, volume, shaping, and, for consumers having curly, coily, or wavy hair, curl definition.

### SUMMARY OF THE DISCLOSURE

The present disclosure therefore relates to hair treatment compositions and methods that provide advantageous effects to hair. It has surprisingly been found that compositions and methods according to the instant disclosure impart a variety of benefits to hair. For example, the compositions and methods provide cosmetic attributes, such as conditioning, softness, moisturization, detangling, smoothness, discipline, and frizz control, to keratin materials, while retaining good curl definition.

In one embodiment, the compositions comprise (a) at least one first cationic compound chosen from esterquat compounds; (b) optionally at least one second cationic compound chosen from cationic surfactants other than esterquat compounds; (c) at least one branched fatty alcohol; and (d) at least one fatty ester.

In an embodiment, the at least one first cationic compound is present in an amount of about 0.1% to about 6%, based on the total weight of the composition. In a further embodiment, the at least one first cationic compound is distearoylethyl dimonium chloride.

In an embodiment, the at least one second cationic compound is chosen from cationic surfactants other than esterquat compounds in an amount ranging from about 0.1% to about 6%, based on the total weight of the composition. In a further embodiment, the at least one second cationic compound is behentrimonium chloride.

In an embodiment, the at least one branched fatty alcohol is chosen from alcohols having from 10 to 30 carbons. In a further embodiment, the at least one branched fatty alcohol is chosen from isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol or mixtures thereof. In a further embodiment, the at least one branched fatty alcohol having from 10 to 30 carbons is present in an amount ranging from about 0.1% to about 3%, based on the total weight of the composition. In a further embodiment, the at least one branched fatty alcohol is 2-octyl-1-dodecanol.

In an embodiment, the at least one fatty ester is present in an amount ranging from about 0.1% to about 3%, based on the total weight of the composition. At room temperature, the at least one fatty ester may be in the form of a liquid or a solid. In a further embodiment, the at least one fatty ester is chosen from sunflower oil, corn oil, soybean oil, marrow oil, grapeseed oil, sesame seed oil, hazelnut oil, apricot oil, macadamia oil, amla oil, castor oil, avocado oil, olive oil, rapeseed oil, coconut oil, wheatgerm oil, sweet almond oil, apricot oil, safflower oil, candlenut oil, camelina oil, tamanu oil, babassu oil, pracaxi oil, jojoba oil, shea butter oil, caprylic/capric triglycerides, or mixtures thereof. In a further embodiment, the at least one fatty ester is coconut oil.

In certain embodiments, the composition further comprises a solvent. In one embodiment, the composition comprises from about 50% to about 95% water, by weight relative to the total weight of the composition.

In certain embodiments, the weight ratio of the total amount of the at least one first cationic compound and the at least one second cationic compound to the total amount of branched fatty alcohol ranges from about 10:1 to about 1:10, from about 5:1 to about 1:5, or from about 3:1 to about 1:3.

In certain embodiments, the weight ratio of the total amount of the at least one first cationic compound and the at least one second cationic compound to the total amount of fatty esters ranges from about 10:1 to about 1:10, from about 5:1 to about 1:5, or from about 3:1 to about 1:3

The composition can be substantially free of synthetic gums, silicones, and/or synthetic polymers. In certain embodiments, at least 0.1% of the materials used in the composition are plant-based and/or of plant origin.

Another embodiment involves a method for treating hair, said method comprising: (1) applying applying to the hair a composition comprising: (a) at least one first cationic compound chosen from esterquat compounds; (b) optionally at least one second cationic compound chosen from cationic surfactants other than esterquat compounds; (c) at least one branched fatty alcohol; and (d) at least one fatty ester, and (2) rinsing the hair. In certain embodiments, step (1) is conducted after treating the hair with a shampoo.

### BRIEF DESCRIPTION OF THE DRAWINGS

The present disclosure and claims can be better understood from the following detailed description either alone or together with the accompanying drawings. The drawings are included to provide a further understanding and are incorporated in and constitute a part of this specification. The drawings illustrate one or more exemplary embodiments of the present disclosure and, together with the description, explain various principles and operation.

FIG. 1 shows photographic images of dry wavy hair swatch samples that were treated with the inventive formulation (11) in comparison to dry wavy swatches that were treated with comparative formulations (C1 through C3).

FIG. 2 shows photographic images of dry wavy hair swatch samples that were treated with the inventive formulations (I1 through I5) in comparison to dry wavy swatches that were treated with comparative formulations C4 through C6.

### DETAILED DESCRIPTION

The disclosure relates to compositions and methods for treating, caring for, and/or conditioning keratin fibers, such as hair. The compositions comprise synergistic combinations of cationic compounds and branched fatty alcohols. The compositions also comprise synergistic combinations of cationic compounds and fatty esters. The methods comprise applying the compositions to the hair.

### I. Compositions

The compositions according to the disclosure comprise synergistic combinations of cationic compounds and branched fatty alcohols, and synergistic combinations of cationic compounds and fatty esters, which separately or in combination surprisingly and unexpectedly lead to improved properties. The compositions may, for example, be hair treatment, hair care, and/or hair conditioning compositions.

### Cationic Compounds

Compositions according to the disclosure comprise (a) at least one first cationic compound chosen from esterquat compounds, and optionally (b) at least one second cationic compound chosen from cationic surfactants other than esterquat compounds.

The total amount of the at least one first cationic compound chosen from esterquat compounds may vary, but typically ranges from about 0.001% to about 20%, about 0.01% to about 15%, or about 0.05% to about 10%, based on the total weight of the composition. For example, the total amount of the at least one first cationic compound chosen from esterquat compounds may range from about 0.01% to about 10%, such as from about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, about 0.05% to about 10%, about 0.05% to about 9%, about 0.05% to about 8%, about 0.05% to about 7%, about 0.05% to about 6%, about 0.05% to about 5%, about 0.05% to about 4%, about 0.05% to about 3%, about 0.05% to about 2%, about 0.05% to about 1%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1%, about 1% to about 10%, about 1% to about 9%, about 1% to about 8%, about 1% to about 7%, about 1% to about 6%, about 1% to about 5%, about 1% to about 4%, about 1% to about 3%, or about 1% to about 2%, including all ranges and sub-ranges there between, based on the total weight of the composition.

The total amount of the optional at least one second cationic compound chosen from cationic surfactants other than esterquat compounds may vary, but typically ranges from about 0.001% to about 10%, such as about 0.01% to about 8%, or about 0.1% to about 6%, based on the total weight of the present composition. For example, the total amount of the at least one second cationic polymer may range from about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, 0.03% to about 8%, about 0.03% to about 7%, about 0.03% to about 6%, about 0.03% to about 5%, about 0.03% to about 4%, about 0.03% to about 3%, about 0.03% to about 2%, about 0.03% to about 1%, 0.05% to about 8%, about 0.05% to about 7%, about 0.05% to about 6%, about 0.05% to about 5%, about 0.05% to about 4%, about 0.05% to about 3%, about 0.05% to about 2%, or about 0.05% to about 1%, about 0.1% to about 8%, about 0.1% to about 7%, about 0.1% to about 6%, about 0.1% to about 5%, about 0.1% to about 4%, about 0.1% to about 3%, about 0.1% to about 2%, or about 0.1% to about 1%, including all ranges and sub-ranges there between, based on the total weight of the composition.

### Cationic Surfactants

In certain embodiments, the at least one first cationic compound and/or the at least one second cationic compound are cationic surfactants that are plant-based and/or organic.

In an embodiment, the at least one first cationic compound is a cationic surfactant chosen from esterquat compounds. As used herein, the term “esterquat compound” means a compound having both at least one ester function and at least one quaternary ammonium group. Exemplary esterquat compounds useful according to the disclosure include quaternized ester salts of fatty acids with triethanolamine, quaternized ester salts of fatty acids with diethanolalkylamines, and quaternized ester salts of fatty acids with 1,2-dihydroxypropyldialkylamines, such as methyl-N-(2-hydroxyethyl)-N,N-di(tallowacyloxyethyl) ammonium compounds, bis-(palmitoyloxyethyl) hydroxyethylmethylammonium compounds, methyl-N,N-bis(stearoyloxyethyl)-N-(2-hydroxyethyl) ammonium compounds, methyl-N,N-bis(cocoyloxyethyl)-N-(2-hydroxyethyl) ammonium compounds, or N,N-dimethyl-N,N-di(tallowacyloxyethyl) ammonium compounds. By way of non-limiting example, esterquat compounds useful according to the disclosure may be chosen from distearoylethyl dimonium chloride, dibehenoylethyl dimonium chloride, dipalmitoylethyl dimonium chloride, ditallowoyl PG-dimonium chloride, dipalmitoylethyl hydroxyethylmonium methosulfate, distearoylethyl hydroxyethylmonium methosulfate, and mixtures thereof.

In one exemplary embodiment, the esterquat compound is distearoylethyl dimonium chloride, which is commercially available under the tradename, “VARISOFT EQ 65 MB,” from the company, Evonik.

The at least one second cationic compound may be chosen from cationic surfactants other than esterquat compounds. By way of non-limiting example, cationic surfactants other than esterquats useful according to the disclosure may be chosen from behentrimonium chloride; cetrimonium chloride; quaternium-22; behenylamidopropyl-2; 3-di-hydroxypropyl-dimethylammonium chloride; palmitylamidopropyltrimethylammonium chloride; oleocetyldimethyl-hydroxyethylammonium chloride; stearamidopropyldimethyl (myristyl acetate) ammonium chloride; di(C1-C2 alkyl) (C12-C22 alkyl)hydroxy(C1-C2alkyl)ammonium salt; alkyltrimethylammonium salt in which the alkyl radical comprises 12 to 24 carbon atoms; propanetallowdiammonium dichloride; behentrimonium methosulfate; chloride and methyl sulfates of diacyloxyethyl-dimethylammonium, of diacyloxyethyl-hydroxyethylmethylammonium, of monoacyloxyethyl-dihydroxyethyl-methylammonium, of triacyloxyethyl-methylammonium, or of monoacyloxyethyl-hydroxyethyl-dimethyl-ammonium; distearyldimethylammonium chloride; behentrimonium methosulfate; or mixtures thereof.

In one exemplary embodiment, the cationic surfactant is chosen from behentrimonium chloride, cetrimonium chloride, behentrimonium methosulfate, and mixtures thereof.

### Branched Fatty Alcohols

The compositions described herein comprise at least one branched fatty alcohol. The at least one branched fatty alcohol may be plant-based and/or organic, or synthetic.

In certain exemplary embodiments, the at least one branched fatty alcohol is neither oxyalkylenated (in particular neither oxyethylenated nor oxypropylenated) nor glycerolated. The at least one branched fatty alcohols can be represented by the formula R—OH, wherein R denotes a saturated (alkyl) or unsaturated (alkenyl) group, branched, and optionally substituted with one or more hydroxyl groups, for example comprising from 8 to 40 carbon atoms, such as from 10 to 30 carbon atoms, from 12 to 24 carbon atoms, or from 14 to 22 carbon atoms.

The at least one branched fatty alcohol may be liquid or solid. The at least one branched solid fatty alcohols that can be used include those that are solid at ambient temperature and at atmospheric pressure (25° C., 780 mmHg) and are insoluble in water, i.e., they have a water solubility of less than 1% by weight, and preferably less than 0.5% by weight, at 25° C. and 1 atm.

The at least one branched solid fatty alcohols may, for example, be represented by the formula R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

In certain embodiments, the at least one branched fatty alcohol, for example those containing from about 10 carbons to about 40 carbons or from about 12 carbons to about 34 carbons, have branched carbon chains and have one or more, such as 1 to 3, double bonds. They are optionally unsaturated (C═C double bond), and for example contain from 12 to 40 carbon atoms.

The at least one branched fatty alcohol may be represented by the formula R—OH, wherein R denotes a C12-C24 branched alkyl group, R being optionally substituted by one or more hydroxy groups. The at least one branched fatty alcohol may be a branched saturated alcohol. Optionally, R does not contain a hydroxyl group.

Non-limiting examples of the at least one branched fatty alcohol includes isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol or mixtures thereof. In one exemplary embodiment, the at least one branched fatty alcohol may be 2-octyl-1-dodecanol.

The total amount of the at least one branched fatty alcohol may vary, but typically ranges from about 0.001% to about 10%, such as from about 0.01% to about 8%, or about 0.1% to about 5%, or about 0.5% to about 3%, based on the total weight of the composition. For example, the total amount of the at least one branched fatty alcohol may range from about 0.01% to about 10%, about 0.01% to about 9%, about 0.01% to about 8%, from about 0.01% to about 7%, from about 0.01% to about 6%, from about 0.01% to about 5%, from about 0.01% to about 4%, about 0.01% to about 3%, about 0.01% to about 2%, about 0.01% to about 1%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1.5%, or from about 0.5% to about 1%, including all ranges and sub-ranges there between, based on the total weight of the composition.

### Fatty Esters

In certain exemplary embodiments, compositions according to the disclosure comprise at least one fatty ester. In some embodiments, the at least one fatty ester is plant-based and/or organic.

In one embodiment, the at least one fatty ester is a liquid ester of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10. In certain embodiments, wherein the at least one fatty ester is an ester of monoalcohols, at least one of the alcohol or of the acid from which the esters of the invention result is branched.

Among the monoesters of monoacids and of monoalcohols, mention may be made of ethyl palmitate, isopropyl palmitate, alkyl myristates such as isopropyl myristate or ethyl myristate, isocetyl stearate, 2-ethylhexyl isononanoate, isononyl isononanoate, isodecyl neopentanoate, isostearyl neopentanoate, and mixtures thereof

Esters of C₄-C₂₂ dicarboxylic or tricarboxylic acids and of C₁-C₂₂ alcohols and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may be made especially of: diethyl sebacate; diisopropyl sebacate; bis(2-ethylhexyl) sebacate; diisopropyl adipate; di-n-propyl adipate; dioctyl adipate; bis(2-ethyhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate; and mixtures thereof.

The composition may also comprise, as fatty ester, sugar esters and diesters of C₆-C₃₀ and preferably C₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars can be monosaccharides, oligosaccharides or polysaccharides.

Mention may be made, as suitable sugars, for example, of sucrose (or saccharose), glucose, galactose, ribose, fucose, maltose, fructose, mannose, arabinose, xylose, lactose and their derivatives, in particular alkyl derivatives, such as methyl derivatives, for example methylglucose, and mixtures thereof.

The esters of sugars and of fatty acids can be chosen in particular from the group of the esters or mixtures of esters of sugars described above and of saturated or unsaturated and linear or branched C₆-C₃₀ fatty acids, and in certain embodiments the C₁₂-C₂₂ fatty acids. If they are unsaturated, these compounds may comprise one to three conjugated or unconjugated carbon-carbon double bonds.

The esters according to this alternative form can also be chosen from mono-di-, tri- and tetraesters, polyesters and their mixtures. These esters can, for example, be oleates, laurates, palmitates, myristates, behenates, cocoates, stearates, linoleates, linolenates, caprates, arachidonates or their mixtures, such as, in particular, oleate/palmitate, oleate/stearate or palmitate/stearate mixed esters. More particularly, use is made of mono- and diesters and in particular mono- or di-oleate, -stearate, -behenate, -oleate/palmitate, -linoleate, -linolenate or -oleate/stearate of sucrose, glucose or methylglucose. Mention may be made, by way of example, of the product sold under the name, “Glucate® DO” by Amerchol, which is a methylglucose dioleate.

Finally, natural or synthetic esters of monoacids, diacids or triacids with glycerol may also be used. Among these, mention may be made of plant oils. As oils of plant origin or synthetic triglycerides that may be used in the composition of the invention as liquid fatty substances, examples that may be mentioned include: triglyceride oils of plant or synthetic origin, such as liquid fatty acid triglycerides containing from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, amla oil, castor oil, avocado oil, camellia oil, camelina oil, olive oil, caprylic/capric acid triglycerides, for instance those sold by the company Stearineries Dubois or those sold under the names “Miglyol® 810”, “Miglyol® 812,” and “Miglyol® 818” by the company, Dynamit Nobel, jojoba oil, rapeseed oil, coconut oil, wheatgerm oil, sweet almond oil, safflower oil, candlenut oil, tamanu oil, babassu oil, pracaxi oil, shea butter oil, and mixtures thereof.

In certain embodiments, liquid fatty esters derived from monoalcohols or triglycerides of plant origin may be used as the fatty esters according to the invention.

According to one embodiment, the fatty esters are chosen from hydrocarbon-based oils comprising more than 6 carbon atoms, such as liquid paraffin; liquid petroleum jelly; esters of C₁-C₂₆ aliphatic mono acids and of C₁-C₂ aliphatic monoalcohols, these esters having a total number of carbon atoms of greater than or equal to 10, in particular isopropyl myristate and isononyl isononanoate; plant oils, in particular coconut oil, shea butter oil, and olive oil; and mixtures thereof.

The total amount of the at least one fatty ester may vary, but typically ranges from about 0.001% to about 10%, such as from about 0.01% to about 5%, or about 0.1% to about 3%, or about 1% to about 2% based on the total weight of the composition. For example, the total amount of the at least one fatty ester may range from about 0.01% to about 10%, from about 0.01% to about 9%, from about 0.01% to about 8%, from about 0.01% to about 7%, from about 0.01% to about 6%, from about 0.01% to about 5%, from about 0.01% to about 4%, from about 0.01% to about 3%, from about 0.01% to about 2%, from about 0.01% to about 1%, from about 0.05% to about 10%, from about 0.05% to about 9%, from about 0.05% to about 8%, from about 0.05% to about 7%, from about 0.05% to about 6%, from about 0.05% to about 5%, from about 0.05% to about 4%, from about 0.05% to about 3%, from about 0.05% to about 2%, from about 0.05% to about 1%, from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1.5%, from about 0.5% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 1% to about 1.5%, including all ranges and sub-ranges there between, based on the total weight of the composition.

### Solvents

In certain exemplary embodiments, compositions according to the disclosure comprise at least one physiologically acceptable medium, i.e., a medium that is compatible with human keratin materials such as the skin (of the body, face, around the eyes or the scalp), the hair, the eyelashes, the eyebrows, bodily hair, the nails, or the lips. The physiologically acceptable medium may be chosen from water or a mixture of water and at least one cosmetically acceptable solvent. Non-limiting examples of cosmetically acceptable solvents include C2-C4 lower alcohols, such as ethanol and isopropanol; polyols, especially those containing from 2 to 6 carbon atoms, for instance glycerol, propylene glycol, butylene glycol, pentylene glycol, hexylene glycol, dipropylene glycol or diethylene glycol; polyol ethers, for instance 2-butoxyethanol, propylene glycol monomethyl ether and diethylene glycol monomethyl ether or monoethyl ether; or mixtures thereof.

In one exemplary embodiment, the composition comprises a solvent or solvent mixture in an amount up to about 99%, such as ranging from about 50% to about 95% or about 60% to about 90%, by weight relative to the weight of the composition. For example, the composition may comprise water in an amount up to about 99%, such as, for example, an amount ranging from about 50 to about 95%, or from about 60% to about 90%, by weight relative to the weight of the composition.

In some embodiments, the weight ratio of the total amount of first and second cationic compounds to the total amount of branched fatty alcohol ranges from about 1:15 to about 15:1, such as from about 1:10 to about 10:1, from about 1:5 to about 5:1, or from about 1:3 to about 3:1. In further embodiments, the weight ratio of the total amount of first and second cationic compounds to the total amount of fatty esters ranges from about 1:15 to about 15:1, such as from about 1:10 to about 10:1, from about 1:5 to about 5:1, or from about 1:3 to about 3:1.

### Additional Components

The compositions according to the disclosure may also comprise additives chosen from nacreous agents, dyes or pigments, fragrances, mineral, plant or synthetic oils, waxes, vitamins, proteins including ceramides, vitamins, UV-screening agents, free-radical scavengers, antidandruff agents, hair-loss counteractants, hair restorers, preserving agents, or mixtures thereof. A person skilled in the art will take care to select the optional additives and the amount thereof such that they do not harm the properties of the compositions of the present disclosure.

The compositions of certain embodiments may comprise stabilizers, for example sodium chloride, magnesium dichloride or magnesium sulfate.

The compositions according to the disclosure may additionally comprise cosmetic adjuvants chosen from fragrances, pigments, chelating agents, softeners, antioxidants, opacifiers, stabilizers, moisturizing agents, vitamins, bactericides, preservatives, polymers, thickening agents, or any other ingredient commonly used in cosmetics for this type of application.

In certain embodiments, these additives are generally present in an amount ranging up to about 40% by weight of active material relative to the total weight of the composition, such as up to about 30%, up to about 20%, up to about 15%, up to about 10%, up to about 5%, such as from 0.01% to 30%.

If desired, a person of skill in the art can select the additives or amounts thereof in order to maintain the natural or organic properties of the compositions.

In some embodiments, the compositions are free or substantially free of secondary gums, silicones, and/or synthetic polymers. For example, the compositions include less than about 3%, less than about 2%, less than about 1%, or less than about 0.5% of secondary gums, silicones, and/or synthetic polymers, and in some embodiments comprise no secondary gums, silicones, and/or synthetic polymers. In some embodiments, however, the compositions may comprise secondary gums, silicones, and/or synthetic polymers. Non-limiting examples of silicones include amine-functionalized silicones (e.g., amodimethicone), dimethicone, bis-aminopropyl dimethicone, and trimethyl silylamodimethicone.

In certain embodiments, at least 85%, at least 88%, at least 90%, or at least 95% of the disclosed materials used in the compositions are plant-based. In one exemplary embodiment, compositions according to the disclosure demonstrate desirable cosmetic properties while comprising mostly, such as at least about 90%, natural origin ingredients.

The compositions described herein may be in any suitable physical form. Suitable forms include, but are not limited to, low to moderate viscosity liquids, lotions, milks, gel creams, creams, pastes, clays, conditioners, masks, and the like. In various embodiments, the compositions may have a lamellar phase structure.

The compositions may be packaged in a variety of different containers, such as, for example, a ready-to-use container. Non-limiting examples of useful packaging include tubes, jars, caps, unit dose packages, and bottles, including squeezable tubes and bottles. In some cases, the packaging is a tube, such as a tube with two compartments, or dual tubes, each forming a separate compartment. Each compartment may include a different composition. For example, one tube or compartment may include a composition according to the instant disclosure, and the other tube may include a composition to be used with the composition, for example, a shampoo or a hair repair or hair treatment product.

The pH of the compositions according to the instant disclosure will vary. Nonetheless, in various embodiments, the hair treatment compositions have a pH of about 3 to about 10. In further embodiments, the hair treatment composition has a pH of about 3 to about 8.5, about 3 to about 8, about 3.5 to about 7, about 3.5 to about 6, about 3.5 to about 5, about 3.5 to about 4.5, or about 3.5 to about 4.

The compositions of the instant disclosure are unique in their ability to provide hair with improved conditioning properties, including but not limited to suppleness, softness, moisturization, conditioning, detangling, smoothness, discipline, and frizz control while maintaining appropriate body, volume, fullness, movement, bounce, and styleability. Accordingly, the instant disclosure relates to methods for treating hair, for example, for improving the manageability of hair and for imparting smoothness. More specifically, the compositions may be used in methods for conditioning the hair, improving ease of combability and detangling, and/or providing smoothness, etc.

### II. Methods

The methods according to the disclosure comprise treating keratinous fibers with the compositions described herein. The methods generally comprise applying any of the disclosed compositions to hair.

By way of example, the methods may include use of the compositions as a conditioner, as part of a shampoo (e.g. a conditioning shampoo), as a pre-treatment, or as a rinse-off mask treatment. In one exemplary embodiment, the composition is a wash-out conditioner. Alternative, in certain embodiments, the hair treatment compositions may be used as a leave-on product. The hair treatment compositions are applied to wet or damp hair and allowed to remain on the hair indefinitely, i.e., the hair treatment composition is not removed or rinsed from the hair prior to drying or styling the hair.

Methods of treating hair according to the disclosure may include applying a composition according to the disclosure to hair (wet, damp, or dry hair), allowing the composition to remain on the hair for a desired amount of time, and optionally rinsing the composition from the hair. The composition may be applied to the hair before, during, or after other hair compositions (e.g., a shampoo, a conditioner, a mask, a cream, a lotion, a gel, etc.).

The composition may be allowed to remain on the hair for a period of time, for example from about a few seconds (e.g. about 10, 20, or 30 seconds) to about 1, 2, 3, 5, 10, 20, or 30 minutes, or longer. If desired, the hair may be heated while the composition is on the hair.

### III. Kits

The hair compositions of the instant disclosure may be incorporated into a kit. For example, the kits may include at least conditioner composition according to the instant disclosure and one or more additional compositions, for example, a shampoo, a hair repair or hair treatment product, etc. The various compositions are separately contained in the kits. In some instances, the kits include one or more compositions according to the instant disclosure and a shampoo, which are separately contained. The kits may also include one or more conditioning compositions according to the instant disclosure, a shampoo, and a hair repair or hair treatment product, all of which are separately contained. Instructions, mixing components, brushes, gloves, measuring tools, etc., may optionally be included in the kits.

It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

The terms “a,” “an,” “the,” and “at least one” are understood to encompass the plural as well as the singular.

Thus, the term “a mixture thereof” also relates to “mixtures thereof.” Throughout the disclosure, if the term “a mixture thereof” is used, following a list of elements as shown in the following example where letters A-F represent the elements: “one or more elements selected from the group consisting of A, B, C, D, E, F, or mixtures thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting.

Some of the compounds discussed throughout the disclosure may be in the form of a salt in the composition or added to the composition in the form of a salt (and dissociate in the composition). Thus, all compounds and amounts of compounds relate to both the salt form of the compound and to the disassociated form of the compound. In other words, even if the expression “a salt thereof” is not specifically or expressly stated with respect to ingredients that can form salts or are available as salts, it is understood that the salt form of the compound is included.

The expression “one or more” means “at least one” and thus includes individual components as well as mixtures/combinations.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

Unless otherwise indicated, all percentages herein are by weight, relative to the weight of the total composition.

The term “surfactants” includes salts of the surfactants even if not explicitly stated

The term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure.

The term “substantially free” or “essentially free” as used herein may also mean that the specific material is not added to the composition but may still be present in a raw material that is included in the composition.

The term “synthetic” means a material that is not of natural origin. The term “natural” means a material of natural origin, such as derived from plants, which also cannot be subsequently chemically or physically modified.

The term “organic” means a material that is produced substantially without or essentially without the use of synthetic materials. The term “substantially without” or “essentially without” as used herein means the specific material may be used in a manufacturing process in small amounts that do not materially affect the basic and novel characteristics of the compositions according to the disclosure. The term “substantially without” or “essentially without” as used herein may also mean that the specific material is not used in a manufacturing process but may still be present in a raw material that is included in the composition.

“Cosmetic composition” encompasses many types of compositions for application to keratin materials such as skin or hair, for example, hair lotions, hair creams, hair gel creams, hair conditioners, hair masques (masks), etc., which can be used either as leave-on or rinse-off treatments or products.

The term “rinse-off” as used herein indicates that the composition is used in a context whereby the composition is ultimately rinsed or washed from the treated surface, (e.g. skin, hair, or hard surfaces) either after or during the application of the product. These rinse-off compositions are to be distinguished from compositions referred to a “leave-on” compositions. For example, a rinse-off composition is applied to the hair, optionally allowed to remain on the hair for a short time (e.g., a few second up to about 5, 10, or 15 minutes) and subsequently rinsed from the hair before the hair styled.

The compositions described throughout this disclosure may be a “leave-on” composition. A “leave-on” (also called leave-in) composition refers to a composition that is applied to skin or hair and is not subjected to immediate rinsing and/or washing, for example for at least 4 hours or for a period of time ranging from 4 hours up to 72 hours, from 4 hours up to 48 hours, or from 8 hours up to 36 hours, or from 8 hours up to 24 hours. In other words, the product is applied to the hair and remains on the hair, as styled, i.e., it is not removed from the hair prior to styling the hair.

A “lamellar phase” refers generally to packing of polar-headed long chain nonpolar-tail molecules in an environment of bulk polar liquid (i.e., water from the hair), as sheets of bilayers separated by bulk liquid.

The term “transparent” with respect to a transparent composition indicates that the composition has transmittance of at least 80% at a wavelength of 600 nm, for example measured using a Lambda 40 UV-visible spectrometer. The compositions may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure. A human can typically see through a transparent composition, for example, and read the text on the other side of a clear glass or clear plastic bottle containing the composition.

The term “translucent” with respect to a translucent composition indicates that the composition has a transmittance of at least 50% at a wavelength of 600 nm, for example measured using a Lambda 40 UV-visible spectrometer. A human cannot likely see through a translucent composition, for example, and read the text on the other side of a clear glass or clear plastic bottle containing the composition. Rather, the text is usually blurred and difficult or not possible to read, yet movement and structure can normally be identified.

The term “opaque” with respect to an opaque composition indicates that the composition is not transparent or translucent, i.e., has a transmittance of less than 50% at a wavelength of 600 nm, for example measured using a Lambda 40 UV-visible spectrometer.

The term, “cosmetically acceptable” means that the item in question is compatible with a keratinous substrate such as skin and hair. For example, a “cosmetically acceptable carrier” means a carrier that is compatible with a keratinous substrate such as skin and hair.

The term “polymers,” as defined herein, include homopolymers and copolymers formed from at least two different types of monomers.

The term “INCI” is an abbreviation of International Nomenclature of Cosmetic Ingredients, which is a system of names provided by the International Nomenclature Committee of the Personal Care Products Council to describe personal care ingredients.

All components and elements positively set forth in this disclosure can be negatively excluded from the claims. In other words, the compositions of the instant disclosure can be free or essentially free of all components and elements positively recited throughout the instant disclosure.

Some of the various categories of components identified may overlap. In such cases where overlap may exist and the composition includes both components (or the composition includes more than two components that overlap), an overlapping component or ingredient does not represent more than one component. In other words, a single compound or ingredient cannot simultaneously serve as two different components of a claimed composition.

It will be apparent to those skilled in the art that various modifications and variations can be made in the compositions and methods according to the disclosure without departing from the spirit or scope of the disclosure. Thus, it is intended that the disclosure in the above description and in the examples given below cover such modifications and variations and their equivalents, shall be interpreted as illustrative and not in a limiting sense.

### EXAMPLES

The following examples are intended to be non-limiting and explanatory in nature only.

### Example 1—Preparation of Conditioner and Shampoo Formulations

Conditioner formulations comprising the following ingredients were prepared and are disclosed in Table 1 below. Amounts are expressed in wt % of active materials:

TABLE 1
Conditioner Formulations

| | Inventive | Comparative | Comparative | Comparative |
|---|---|---|---|---|
| | Conditioner 1 | Conditioner 1 | Conditioner 2 | Conditioner 3 |
| US INCI NAME | (I1) | (C1) | (C2) | (C3) |
| water | qs | qs | qs | qs |
| Distearoylethyl dimonium | 1.43 | 2.275 | 2.249 | — |
| chloride | | | | |
| Behentrimonium chloride | 2.84 | — | — | — |
| Octyldodecanol | 1.00 | — | — | — |
| Cocos nucifera (coconut) oil | 1.50 | — | — | 0.01 |
| Cetearyl Alcohol | 6.77 | 6.225 | 6.231 | 4.50 |
| C15-19 Alkane | — | 1.00 | 0.50 | — |
| Isopropyl myristate | 1.50 | — | — | 2.00 |
| Helianthus annuus | — | — | — | 0.50 |
| (Sunflower) seed oil | | | | |
| Glycine Soja (Soybean) oil | — | — | — | 1.00 |
| Coco-caprylate/Caprate | — | — | — | 0.50 |
| Cetyl esters | — | — | | 0.50 |
| Guar | — | 0.05 | 0.18 | — |
| hydroxypropyltrimonium | | | | |
| chloride | | | | |
| Hydroxypropyl Guar | — | — | — | 0.05 |
| Hydroxypropyltrimonium | | | | |
| chloride | | | | |
| Stearamidopropyl | — | — | — | 1.00 |
| dimethylamine | | | | |
| Glyceryl stearate | — | 1.00 | 1.00 | — |
| Glyceryl caprylate | — | 0.50 | 0.50 | — |
| Hydroxypropyl guar | 0.10 | — | — | — |
| Potato starch modified | 0.215 | — | — | — |
| glycerin | 2.00 | 4.00 | 2.00 | 3.05 |
| sorbitol | 2.00 | — | — | — |
| Hydroxyethyl urea | 1.25 | — | — | — |
| arginine | — | 0.12 | 0.12 | — |
| glycine | 0.10 | — | | — |
| Aloe barbadensis leaf | — | 0.08 | 0.08 | — |
| juice powder | | | | |
| Miscellaneous ingredients* | ≤5 | ≤5 | ≤5 | ≤5 |
| (preservatives, fragrances, | | | | |
| pH adjusters, etc.) | | | | |
| Translucent/transparent/opaque | Opaque | Opaque | Opaque | Opaque |
| pH | 3.8 | 4.2 | 4.0 | 3.7 |
| Viscosity cP (measured at | 22,000 | 29,000 | 17,500 | 20,000 |
| 25° C. measured with a | | | | |
| Brookfield DV-LL+ Pro | | | | |
| Viscometer using Helipas | | | | |
| 94/D spindles and rotational | | | | |
| speed of 20% RPM) | | | | |

pH adjusters such as citric acid, tartaric acid, sodium hydroxide, and sodium citrate; preservatives such as salicylic acid, phenoxyethanol, benzoic acid, benzyl alcohol, and caprylyl glycol)

A shampoo formulation comprising the following ingredients was prepared and is disclosed in Table 2 below. Amounts are expressed in wt % of active materials:

TABLE 2
Shampoo Formulation

| | Cosmetic | Wt % of |
|---|---|---|
| US INCI NAME | type | active(s) |
| LAURETH-12; COCO-BETAINE; | SURFACTANT | 12.15 |
| COCAMIDEMEA; SODIUM LAURETH | | |
| SULFATE | | |
| AQUA | SOLVENT | QS |
| PROPYLENE GLYCOL; HEXYLENE | SOLVENT | 3.68 |
| GLYCOL; GLYCERIN ONE OR | | |
| MORE OF: DYES, PRESERVATIVES, | ADDITIVES | ≤5 |
| FRAGRANCE, FLOWER EXTRACTS | | |

Inventive conditioner formulations (11 to 15) and comparative conditioner formulations (04-06) comprising the following ingredients were prepared and are disclosed in Table 3A and Table 3B below. All amounts are expressed in wt % of active materials:

TABLE 3A
Conditioner Formulations

| INCI NAME | (I1) | (C5) | (I3) | (I4) | (I5) | (I2) | (C4) | (C6) |
|---|---|---|---|---|---|---|---|---|
| ISOPROPYL MYRISTATE | 1.50 | 1.50 | 1.50 | 1.50 | 1.50 | 1.50 | 1.50 | 1.50 |
| HYDROXYETHYL UREA | 1.23 | 1.23 | 1.23 | 1.23 | 1.23 | 1.23 | 1.23 | 1.23 |
| POTATO STARCH MODIFIED | 0.22 | 0.22 | 0.22 | 0.22 | 0.22 | 0.22 | 0.22 | 0.22 |
| BUTYROSPERMUM PARKII (SHEA) | | | 1.50 | | | | | |
| BUTTER | | | | | | | | |
| HYDROXYPROPYL GUAR | 0.10 | 0.10 | 0.10 | 0.10 | 0.10 | 0.10 | 0.10 | 0.10 |
| SORBITOL | 2.00 | 2.00 | 2.00 | 2.00 | 2.00 | 2.00 | 2.00 | 2.00 |
| OCTYLDODECANOL | 1.00 | | 1.00 | 1.00 | 1.00 | 1.00 | 1.00 | 1.00 |
| GLYCINE | 0.10 | 0.10 | 0.10 | 0.10 | 0.10 | 0.10 | 0.10 | 0.10 |
| GLYCERIN | 2.00 | 2.00 | 2.00 | 2.00 | 2.00 | 2.00 | 2.00 | 2.00 |
| CETEARYL ALCOHOL | 6.77 | 6.77 | 6.77 | 6.77 | 6.77 | 6.77 | 6.00 | 6.77 |
| DISTEAROYLETHYL DIMONIUM | 1.43 | 1.43 | 1.43 | 1.43 | 1.43 | 1.43 | | 1.43 |
| CHLORIDE | | | | | | | | |
| BEHENTRIMONIUM CHLORIDE | 2.84 | 2.84 | 2.84 | 2.84 | 2.84 | | 2.84 | 2.84 |
| WATER | qs | qs | qs | qs | qs | qs | qs | sb |
| OLEA EUROPAEA (OLIVE) FRUIT OIL | | | | | 1.50 | | | |
| COCOS NUCIFERA (COCONUT) OIL | 1.50 | 1.50 | | | | 1.50 | 1.50 | |
| SIMMONDSIA CHINENSIS (JOJOBA) | | | | 1.50 | | | | |
| SEED OIL | | | | | | | | |
| One or more preservatives, fragrance, pH | ≤5 | ≤5 | ≤5 | ≤5 | ≤5 | ≤5 | ≤5 | ≤5 |
| adjuster such as sodium citrate, citric acid. | | | | | | | | |

TABLE 3B
Properties of Formulations

| | Inv. | Inv. | Comp. | Comp. | Comp. | Inv. | Inv. | Inv. |
|---|---|---|---|---|---|---|---|---|
| | Cond. | Cond. | Cond. | Cond. | Cond. # | Cond. | Cond. | Cond. |
| | #1 (I1) | #2 (I2) | #4 (C4) | #5 (C5) | 6 (C6) | #3 (I3) | # 4 (I4) | #5 (I5) |
| Translucent/Transparent/ | Opaque | Opaque | Opaque | Opaque | Opaque | Opaque | Opaque | Opaque |
| Opaque | | | | | | | | |
| pH | 3.8 | 3.9 | 3.9 | 3.8 | 3.8 | 3.8 | 3.8 | 3.8 |
| Viscosity cP (measured at | 22,000 | 7,000 | 15,000 | 22,500 | 24,000 | 24,500 | 27,000 | 29,000 |
| 25° C. measured with a | | | | | | | | |
| Brookfield DV-LL + Pro | | | | | | | | |
| Viscometer using Helipas | | | | | | | | |
| 94/D spindles and rotational | | | | | | | | |
| speed of 20% RPM) | | | | | | | | |

### Example 2—General Procedure to Test the Performance Attributes of the Invention on Wavy Hair

The following tests were performed on 11 (CP4) hair swatch samples made of bleached Caucasian curly hair (approximately 2.7 g/swatch in weight and 20 cm/swatch in length when straight).

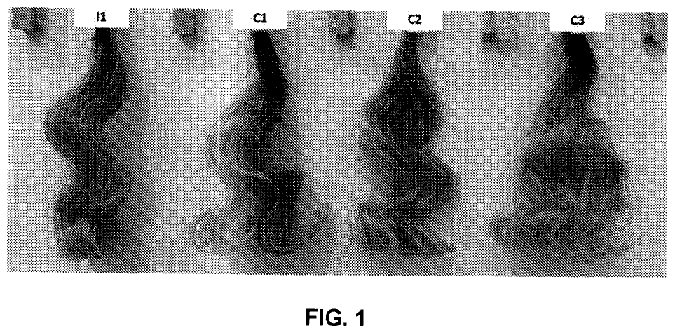

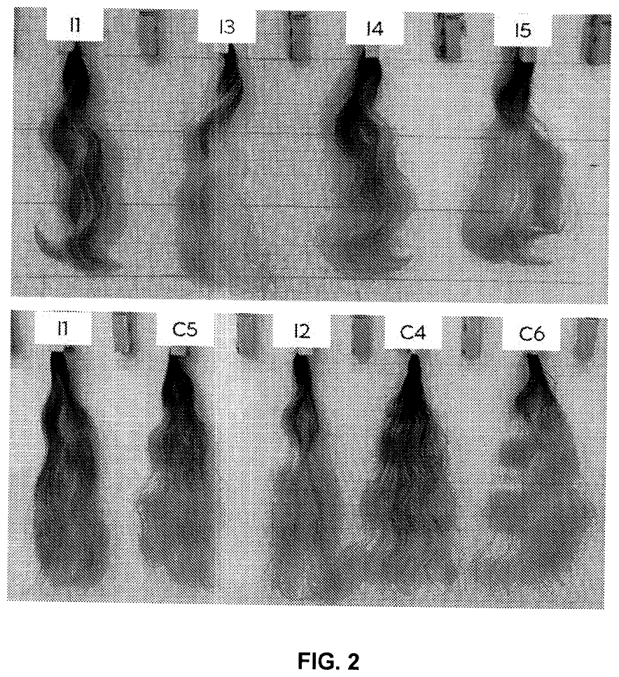

On each swatch sample, the shampoo of Table 2 was applied, lathered into the locks for approximately 10 to 15 seconds, and then rinsed off with water. Each swatch sample was then treated with one of the conditioner formulations set forth in Table 1 and Table 3A. After massaging one of the conditioner formulations into the locks of a swatch for approximately 10 to 15 seconds, the conditioner formulation was rinsed off with water. Excess water was removed from the swatch samples using a towel. For each of the 4 swatch samples (to which Inventive #1 and Comparative #1, #2, and #3 conditioner formulations were applied, respectively), the following performance attributes were measured by three laboratory experts (N=3): wet combing and wet smoothness, and the metrics were based on the average score of their assessment as shown in Table 4A. The swatch samples were dried naturally under ambient conditions before making additional observations. For each of the 11 swatch samples, the following performance attributes were then measured by three laboratory experts (N=3): dry smoothness, dry moisture feel, dry curl definition, and dry anti-frizz. The metrics were based upon the average score of their assessment as shown in Table 4A and Table 4B. Photographs of the dried hair swatch samples (to which Inventive #1 formulation and Comparative #1, #2, and #3 conditioner formulations were applied, respectively) are shown in FIG. 1, and photographs of the dried hair swatch samples (to which Inventive #1 through #5 conditioner formulations and Comparative #4 through #6 conditioner formulations were applied, respectively) are shown in FIG. 2.

TABLE 4A
Wet and Dry Performance Attributes*** of Inventive Formulation and
Comparative Formulations A, B, and C

| Performance | Inv. 1 | Comp. 1 | Comp. 2 | Comp. 3 |
|---|---|---|---|---|
| attribute | (I1) | (C1) | (C2) | (C3) |
| Wet combing | B* | 5 | 5 | 5 |
| Wet Smoothness | | 4 | 5 | 5 |
| Dry Smoothness | | 4 | 3 | 4 |
| Dry Moisture feel | | 5 | 4 | 5 |
| Dry Curl Definition | | 4 | 4 | 5 |
| Dry Anti-frizz | | 5 | 5 | 5 |
| *B = benchmark | | | | |
| ***Sensorial | | | | |
| metrics for the | | | | |
| evaluation of hair | | | | |
| cosmeticity: | | | | |
| 1: Better than | | | | |
| the benchmark | | | | |
| 2: Slightly | | | | |
| better than the | | | | |
| benchmark | | | | |
| 3: Same as the | | | | |
| benchmark | | | | |
| 4: Slightly | | | | |
| inferior than | | | | |
| the benchmark | | | | |
| 5: Inferior than | | | | |
| the benchmark | | | | |

TABLE 4B
Dry Performance Attributes*** of Inventive Formulation
1 through 5 and Comparative Formulations 4 through 6.

| | Inv. | Inv. | Comp. | Comp. | Comp. | Inv. | Inv. | Inv. |
|---|---|---|---|---|---|---|---|---|
| | Cond. | Cond. | Cond. | Cond. | Cond. | Cond. | Cond. | Cond. |
| Performance Attribute | 1 (I1) | 2 (I2) | 4 (C4) | 5 (C5) | 6 (C6) | 3 (I3) | 4 (I4) | 5 (I5) |
| Dry Smoothness | B* | 2 | 4 | 4 | 4 | 4 | 3 | 2 |
| Dry moisture Feel | B* | 4 | 4 | 4 | 5 | 3 | 3 | 4 |
| Dry Curl Definition | B* | 4 | 1 | 2 | 1 | 4 | 4 | 4 |
| Dry Anti-Frizz | B* | 3 | 5 | 4 | 5 | 3 | 4 | 4 |

*B = Benchmark
***= Sensorial metrics for the evaluation of hair cosmeticity:
1: Better than the benchmark
2: Slightly better than the benchmark
3: Same as the benchmark
4: Slightly inferior than the benchmark
5: Inferior than benchmark

This Example showed that the Inventive conditioner formulations achieved the most balanced performance regarding wet and dry hair tactile and visual ques. It was found that the Inventive conditioner formulations unexpectedly imparted conditioning properties to the hair locks including suppleness, softness, moisturization, conditioning, detangling, smoothness, discipline, frizz control, and other cosmetic benefits to keratin materials, while maintaining appropriate body/volume/fullness, movement, bounce and styleability.

1. A composition comprising:
(a) at least one first cationic compound chosen from esterquat compounds;
(b) optionally at least one second cationic compound chosen from cationic surfactants other than esterquat compounds;
(c) at least one branched fatty alcohol; and
(d) at least one fatty ester.

2. The composition of claim 1, wherein the at least one first cationic compound is present in an amount ranging from about 0.1% to about 6%, based on the total weight of the composition.

3. The composition of claim 2, wherein the at least one first cationic compound is present in an amount ranging from about 1% to about 3%, based on the total weight of the composition.

4. The composition of claim 1, wherein the at least one first cationic compound is distearoylethyl dimonium chloride.

5. The composition of claim 1, wherein the at least one second cationic compound is present in an amount ranging from about 0.1 to about 6%, based on the total weight of the composition.

6. The composition of claim 1, wherein the at least one second cationic compound is present in an amount ranging from about 2 to about 4%, based on the total weight of the composition.

7. The composition of claim 1, wherein the at least one second cationic compound is behentrimonium chloride.

8. The composition of claim 1, wherein the at least one branched fatty alcohol is chosen from isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol, or mixtures thereof.

9. The composition of claim 8, wherein the at least one branched fatty alcohol is 2-octyl-1-dodecanol.

10. The composition of claim 8, where in the at least one branched fatty alcohol is present in an amount ranging from about 0.1% to about 3%.

11. The composition of claim 1, wherein the at least one fatty ester is chosen heptanoic or octanoic acid triglycerides, sunflower oil, corn oil, soybean oil, marrow oil, grapeseed oil, sesame seed oil, hazelnut oil, apricot oil, macadamia oil, amla oil, castor oil, avocado oil, camellia oil, camelina oil, olive oil caprylic/capric acid triglycerides, jojoba oil, rapeseed oil coconut oil wheatgerm oil, sweet almond oil, safflower oil, candlenut oil, tamanu oil, babassu oil, pracaxi oil, and shea butter oil, or mixtures thereof.

12. The composition of claim 11, wherein the at least one fatty ester is chosen from coconut oil.

13. The composition of claim 1, wherein the weight ratio of the total amount of the at least one first cationic compound and the at least one second cationic compound to the total amount of the branched fatty alcohol ranges from about 10:1 to about 1:10.

14. The composition of claim 13, wherein the weight ratio of the total amount of the at least one first cationic compound and the at least one second cationic compound to the total amount of the branched fatty alcohol ranges from about 5:1 to about 1:5.

15. The composition of claim 1, wherein the weight ratio of the total amount of the at least one first cationic compound and the at least one second cationic compound to the total amount of the fatty esters ranges from about 10:1 to about 1:10.

16. The composition of claim 1, wherein the weight ratio of the total amount of the at least one first cationic compound and the at least one second cationic compound to the total amount of the fatty esters ranges from about 5:1 to about 1:5.

17. A composition comprising, based upon the total weight of the composition:
(a) from about 1 to about 3% of distearoylethyl dimonium chloride;
(b) from about 2 to about 4% of behentrimonium chloride;
(c) from about 1 to about 2% of 2-octyl-1-dodecanol; and
(d) from about 1 to about 2% of at least one fatty ester chosen from olive oil, coconut oil, and shea butter oil, wherein the weight ratio of distearoylethyl dimonium chloride and behentrimonium chloride to 2-octyl-1-dodecanol is about 10:1 to about 1:10 about 20:1 to about 1:20.

18. The composition of claim 17, wherein the at least one fatty ester is coconut oil.

19. A method for treating hair, said method comprising:
(1) applying to the hair a composition comprising:
at least one first cationic compound chosen from esterquat compounds;
optionally at least one second cationic compound chosen from cationic surfactants other than esterquat compounds;
at least one branched fatty alcohol; and
at least one fatty ester; and
(2) rinsing the hair.

20. The method of claim 19, wherein step (1) is conducted after treating the hair with a shampoo.
